(19) United States
(12) Patent Application Publication
(10) Pub. No.: US 2025/0129327 A1
(43) Pub. Date: Apr. 24, 2025

(54) APPLICATION OF BLAUTIA SP B2132 STRAIN IN PREVENTING AND/OR TREATING INFLAMMATORY BOWEL DISEASE

(71) Applicant: Zhejiang university, Hangzhou (CN)

(72) Inventors: Zhengping XU, Hangzhou (CN); Desen SUN, Hangzhou (CN); Jinghao SHENG, Hangzhou (CN); Rongpan BAI, Hangzhou (CN)

(21) Appl. No.: 17/595,833

(22) PCT Filed: Jan. 8, 2021
(86) PCT No.: PCT/CN2021/070865

§ 371 Date: Nov. 24, 2021

(30) Foreign Application Priority Data
Feb. 24, 2020 (CN) 202010110327.X

(51) Int. Cl. C12N 1/20; A61K 35/741; C12R 1/01

(52) U.S. Cl. CPC C12N 1/205; A61K 35/741; C12R 2001/01

(57) ABSTRACT

Provided are a Blautia sp. B2132 strain and an application thereof. The Blautia sp. B2132 strain has a deposit number of CGMCC No. 1.5296 and is used for regulating the homeostasis of intestinal flora and for preventing or treating the occurrence of an intestinal disease such as an inflammatory bowel disease.

### REFERENCE TO SEQUENCE LISTING

The substitute sequence listing is submitted as an ASCII formatted text filed via EFS-Web, with a file name of “Substitute_Sequence_Listing. TXT”, a creation date of Sep. 7, 2024 and a size of 1398 bytes. The substitute sequence Listing filed via EFS-Web is a part of the specification and is incorporated in its entirety by reference herein.

### TECHNICAL FIELD

The present disclosure belongs to the technical field of microorganisms, specifically relates to an application of Blautia sp. strain in regulating the intestinal flora and preventing and/or treating an inflammatory bowel disease.

### BACKGROUND

Inflammatory bowel disease (IBD) is a class of diseases characterized by chronic inflammation of the digestive tract, mainly including subtypes of ulcerative colitis (UC) and Crohn's disease (CD). The clinical manifestations of IBD are mainly continuous diarrhea, abdominal pain, rectal bleeding/hematochezia, weight loss, etc., which seriously affect the patient's quality of life and are prone to relapse, so it is considered as a lifelong disease. In the past, the main treatment object of ulcerative colitis is to induce or maintain ulcerative colitis in remission through the use of aminosalicylic acid anti-inflammatory drugs, immunosuppressants or colectomy, however, related complications gradually increase after using the traditional treatment protocols.

Intestinal flora plays an essential role in maintaining the homeostasis of intestinal environment. Normal intestinal flora plays an important role in terms of defense against pathogen invasion, synthesis of vitamins, material metabolism, growth and aging, anti-cancer, etc. In recent years, the role of intestinal flora in the occurrence and development of IBD is getting more and more attention. The study has found that compared with the intestinal flora of healthy people, the intestinal flora of IBD patients has changed significantly, and the diversity of its species is significantly lower than that of healthy people. Among them, the diversity of symbiotic anaerobic bacteria Firmicutes and Bacteroidetes decreases significantly, while the diversity of Proteobacteria increases significantly. Analysis of flora composition showed that, the content of Lachnospiraceae in the feces of IBD patients was significantly reduced; while Bifidobacterium breve and Clostridium symbiosum were significantly enriched in UC patients, and 12 kinds of bacteria, including Clostridium clostridioforme, Escherichia coli and the like, increased specifically in CD patients. At the same time, in the Spanish IBD patient cohort study, it was found that 6 kinds of microorganisms, including Faecalibacterium, Peptostreptococcaceae and the like, were significantly reduced in the feces of CD patients, while Fusobacterium and Escherichia were significantly increased. In summary, a large number of clinical data indicated that the intestinal flora of IBD patients is seriously imbalanced.

Based on the fact that intestinal flora imbalance and intraluminal antigen stimulation are important reasons for the onset and recurrence of IBD, probiotics have been widely used in the past 10 years to improve the intestinal microenvironment, restore the body's normal flora, and reduce inflammation, so as to achieve the purposes of controlling the intestinal tract inflammation and maintaining remission, for example, Mesalazine combined with Bifidobacterium has achieved good results in the clinical treatment of IBD. The development of probiotics with different functions is considered to be an important way to prevent and treat IBD in the future. The abundance of Lachnospiraceae in IBD patients is usually significantly lower than that in normal people. However, up to now, there is still a lack of Lachnospiraceae species for the treatment of IBD, so it is of great significance to find and develop Lachnospiraceae strains.

At present, many evidences show that the genus Blautia is closely related to human health, and its abundance imbalance is related to a variety of diseases. The existing Blautia-related inventions are mainly as follows:

The invention “Application of Blautia in the preparation of a product for enhancing the efficacy of hypolipidemic drugs in the treatment of hyperlipidemia” with Application No. 201910901571.5 notifies that: an application of a preparation for improving the level, identity or proportion of Blautia bacteria in the intestinal flora of a subject in the preparation of a product for enhancing the efficacy of hypolipidemic drugs in the treatment of hyperlipidemia, that is, a pharmaceutical composition and an application of the pharmaceutical composition in the preparation of a product for treating hyperlipidemia. By administering to the subject a preparation for improving the level, identity or proportion of Blautia bacteria in the intestinal flora of the subject, the level, identity or proportion of beneficial microorganisms in the subject's body is increased, and the responsiveness of the subject to the lipid-lowering therapy is further enhanced, thereby significantly enhancing the lipid-lowering effect of the hypolipidemic drugs and reducing individual differences.

The invention “Bifidobacterium adolescentis and use thereof” with Application No. 2017109634415 notifies that: Bifidobacterium adolescentis CCFM8630 can significantly increase the level of the neurotransmitter 5-serotonin in the peripheral blood of rats; restore the increase in the level of hormones such as testosterone in the peripheral blood of the rat and the abnormal abundance of the genus Blautia and Turicibacter in the intestinal flora of the rat caused by the high-sugar and high-fat diet; be tolerant to simulated gastrointestinal fluid, rapidly colonize in the intestine, significantly improve metabolic syndromes and pathological injuries of liver and duodenum in rats caused by the high-sugar and high-fat diet as well as the elevated serum triglyceride and total cholesterol levels and oral glucose tolerance, so it can be used for preventing, alleviating or treating metabolic disorders such as metabolic syndrome, irritable bowel syndrome, as well as anxiety, depression and other psychological diseases related to the metabolic syndrome.

The invention “Compound probiotic preparation for preventing and treating piglet diarrhea and preparation method and application thereof” with Application No. 201710117267.2 notifies that: the compound probiotic preparation includes 5 kinds of probiotics, which are Lactobacillus frumenti, Lactobacillus gasseri LA39, Butyricicoccus pullicaecorum, Eubacterium hallii and Blautia hansenii, respectively. The prepared five bacterial suspensions are centrifuged respectively to obtain bacterial precipitates, the obtained bacterial precipitates are resuspended with phosphate buffered saline PBS, and the obtained five viable bacteria suspensions are mixed evenly to get a compound probiotic preparation; the total number of viable bacteria per milliliter of the compound probiotic preparation is 10¹ to 10²⁰. The preparation process of the probiotic preparation is simple, the production cost is low, and it has a significant effect of preventing and treating piglet diarrhea.

The invention “Lactobacillus reuteri and use thereof” with Application No. 2017109631192 notifies that: Lactobacillus reuteri can significantly increase the level of the neurotransmitter 5-serotonin in the peripheral blood of rats; restore the increase in the level of hormones such as testosterone in the peripheral blood of the rat and the abnormal abundance of the genus Blautia, Turicibacter, Oscillospira and Bifidobacterium in the intestinal flora of the rat caused by the high-sugar and high-fat diet; be tolerant to simulated gastrointestinal fluid, rapidly colonize in the intestine, significantly improve metabolic syndromes and pathological injuries of liver and duodenum in rats caused by the high-sugar and high-fat diet as well as the elevated serum triglyceride and total cholesterol levels, so it can be used for preventing, alleviating or treating metabolic disorders such as metabolic syndrome, irritable bowel syndrome, as well as anxiety, depression and other psychological diseases related to the metabolic syndrome.

### SUMMARY

The technical problem to be solved by the present disclosure is to provide an application of Blautia sp. strain in regulating the intestinal flora and preventing and/or treating an inflammatory bowel disease.

That is, the present disclosure provides an intestinal isolated bacterium (Spirillaceae strain), as a probiotic preparation, which is applied in a pharmaceutical composition, food, health care product, food additive and other products for regulating the homeostasis of intestinal flora, preventing and treating the occurrence of intestinal diseases such as an inflammatory bowel disease.

To solve the above technical problem, the present disclosure provides a Blautia sp. B2132 strain, which has a deposit number of CGMCC NO. 1.5296.

The present disclosure also provides an application of the Blautia sp. B2132 strain in the preparation of a medicine for regulating the intestinal flora and preventing and/or treating an inflammatory bowel disease.

As an improvement of the application of the present disclosure: the medicine comprises a pharmaceutically effective dose of Blautia sp. B2132 strain and a pharmaceutically acceptable carrier.

As a further improvement of the application of the present disclosure: the pharmaceutically effective dose is 10⁶-10¹⁰ CFU.

As a further improvement of the application of the present disclosure: the pharmaceutically acceptable carrier is milk powder, lactose, cyclodextrin, maltose, glucose, glycerin, sodium glutamate, vitamin C, mannose, galactose, mannitol or methylcellulose.

As a further improvement of the application of the present disclosure: the inflammatory bowel disease is ulcerative colitis or Crohn's disease.

The present disclosure provides a pharmaceutical composition for regulating the intestinal flora and preventing and/or treating an inflammatory bowel disease, wherein, the pharmaceutical composition comprises a pharmaceutically effective dose of Blautia sp. B2132 strain which has a deposit number of CGMCC No. 1.5296.

As an improvement of the pharmaceutical composition of the present disclosure: the pharmaceutically effective dose is 10⁶-10¹⁰ CFU.

The present disclosure also provides a food/health care product/food additive for preventing and/or treating an inflammatory bowel disease, wherein the food/health care product/food additive comprises a Blautia sp. B2132 strain which has a deposit number of CGMCC No. 1.5296.

The present disclosure provides the separation and identification of Lachnospiraceae Blautia sp. B2132 from feces, of which the preservation information is as follows: the preservation name is Blautia sp., Depository Authority: Common Microorganism Center of China Committee for Culture Collection of Microorganisms, Preservation address: No. 3, Courtyard 1, Beichen West Road, Chaoyang District, Beijing; Deposit No: CGMCC NO. 1.5296, Preservation time: Oct. 8, 2019.

The 16S rDNA partial sequence of Blautia sp. B2132 strain and the morphology of the bacteria observed by a scanning electron microscope are shown in FIG. 1.

The present disclosure also provides a quantitative PCR method for detecting Blautia sp. strain.

The present disclosure has found through detection that the content of Blautia sp. strain in IBD patients decreased significantly, and it was found in the present disclosure that the abundance of Blautia sp. strain in the intestinal flora was negatively correlated with the incidence of enteritis; the Blautia sp. B2132 strain of the present disclosure can inhibit the growth of harmful bacteria in the intestinal tract and regulate the intestinal flora.

The present disclosure also proves that the Blautia sp. B2132 strain has a good effect in preventing and/or treating an inflammatory bowel disease.

The present disclosure has confirmed through clinical samples, animal in vivo and in vitro related experiments that, Blautia sp. B2132 strain can inhibit the growth of harmful intestinal bacteria, improve the intestinal flora, has excellent resistance to inflammatory bowel diseases, including ulcerative colitis or Crohn's disease, and has no toxic side effects, so it can be durably and effectively applied in the preparation of medicines, pharmaceutical compositions, food, health care products or food additives for preventing and/or treating inflammatory bowel diseases. These medicines, pharmaceutical compositions, food, health care products or food additives can be used for preventing and treating inflammatory bowel diseases, and have significant application value.

However, currently there have been no existing technologies mentioning that Blautia sp. has the functions of regulating the intestinal flora, preventing and/or treating inflammatory bowel diseases.

### BRIEF DESCRIPTION OF THE DRAWINGS

The specific embodiments of the present disclosure will be described in further detail below in conjunction with the accompanying drawings.

FIG. 1 shows the identification results of Blautia sp. B2132 strain; wherein,

(A) shows the partial 16S rDNA sequence of Blautia sp. B2132 strain;

(B) shows the phylogenetic classification results of the strain;

(C) shows the morphology of the bacteria observed under a scanning electron microscope.

FIG. 2 shows the abundance detection of Blautia sp. B2132 strain in the fecal flora of healthy controls and IBD patients.

FIG. 3 shows the effect of the abundance of Blautia sp. strain in the intestinal flora on spontaneous enteritis in Il-10^{-/-} mice.

(A) Changes in body weight of Il-10^{-/-} mice with high and low Blautia sp. strain abundance during the spontaneous enteritis;

(B) Length of the colorectum of Il-10^{-/-} mice with high and low Blautia sp. strain abundance at the 85^{th} day of age;

(C) Intestinal H&E staining of Il-10^{-/-} mice with high and low Blautia sp. strain abundance shows the infiltration of intestinal epithelial inflammatory cells;

(D) Expression of inflammatory factors IL-1β, IL-6 and IL-17A in the colon tissue of Il-10^{-/-} mice with high and low Blautia sp. strain abundance.

FIG. 4 shows that Blautia sp. B2132 strain can inhibit the growth of the harmful intestinal bacteria α-proteobacteria.

FIG. 5 shows that oral supplementation of Blautia sp. B2132 strain can increase the abundance of Blautia sp. B2132 strain in the intestinal flora, thereby reducing the phenotype of spontaneous enteritis in Il-10^{-/-} mice; wherein,

(A) shows the body weight change curves of Il-10^{-/-} mice in the BHI control treatment group and Blautia sp. B2132 treatment group during spontaneous inflammation;

(B) shows the length of the colorectum of Il-10^{-/-} mice in the BHI control treatment group and Blautia sp. B2132 treatment group at the 85^{th} day of age;

(C) shows that distal colon H&E staining of Il-10^{-/-} mice in the BHI control treatment group and Blautia sp. B2132 treatment group shows the infiltration of intestinal epithelial inflammatory cells;

(D) shows the expression of inflammatory factors IL-1β, IL-6 and IL-17A in the colon tissue of Il-10^{-/-} mice in the BHI control treatment group and Blautia sp. B2132 treatment group.

### DETAILED DESCRIPTION OF THE EMBODIMENTS

The present disclosure will be further described below in conjunction with specific embodiments, but the protection scope of the present disclosure is not limited to this:

Reagents, Materials and Equipment

| Name | Manufacturer |
|---|---|
| PCR super MIX | Beijing TransGen Biotech Co., Ltd. |
| SYBR ® Premix Ex Taq ™ | Takara Bio Inc., Japan |
| BHI Culture solution | BD Difco Co., USA |
| L-cysteine | Shanghai Aladdin Biochemical |
| | Technology Co., Ltd. |
| Il-10^{-/-} mice | Jackson Lab Co., USA |
| C57/BL6 mice | Shanghai SLAC Laboratory Animal |
| | Co., Ltd. |
| Biochemical incubator | Ningbo Saifu Laboratory Instrument |
| | Factory |
| Anaerobic incubator | Shanghai Longyue Instrument |
| | Equipment Co., Ltd. |
| Clean bench | Suzhou Antai Air Tech Co., Ltd. |
| Quantitative PCR Instrument | Roche Co., USA |
| Scanning electron microscope | FEI Co., USA |

### Embodiment 1. Isolation, Cultivation and Identification of Blautia Sp. B2132 Strain

### (1) Preparation of Conditioned Medium

Pre-deoxygenated liquid BHI medium (containing 5% bovine serum and 0.1% cysteine): Weighing 37 g brain heart infusion (BD Co.) into a 1 L conical flask, adding 1 g cysteine and double distilled water to make the volume to 1000 ml, stirring to dissolve, and then sterilizing in an autoclave at 121° C. for 15 minutes; after sterilization, placing it into an anaerobic incubator, cooling to about 40-50° C., adding 50 ml fetal bovine serum and shaking evenly to obtain pre-deoxygenated liquid BHI medium, and then dispensing into 50 ml centrifuge tubes.

Pre-deoxygenated solid BHI medium (containing 5% bovine serum, 0.1% cysteine and 1 mg/L aztreonam): Weighing 37 g brain heart infusion (BD Co.) into a 1 L conical flask, adding 1 g cysteine, 15 g agar powder and double distilled water to make the volume to 1000 ml, stirring to dissolve, and then sterilizing in an autoclave at 121° C. for 15 minutes; after sterilization, placing it into an anaerobic incubator, cooling to about 40-50° C., adding 50 ml fetal bovine serum and 1 mg aztreonam and shaking evenly to obtain pre-deoxygenated solid BHI medium; then carrying out the inverted plate operation, adding 20-25 ml pre-deoxygenated solid BHI medium into each petri dish with a diameter of 10 cm, and letting it solidify by natural cooling.

### (2) Sample Collection and Bacterial Culture

First, taking 0.1 g fresh healthy mouse feces into a 1.5 ml centrifuge tube, and promptly transferring into an anaerobic incubator, adding 1 ml pre-deoxygenated liquid BHI medium, stirring with a sterile pipette tip into a homogenate; then diluting the bacterial suspension 5 times in a gradient of 1/10 (diluting with the pre-deoxygenated liquid BHI medium as the solvent), spreading 100 μL on the pre-deoxygenated BHI solid plate (pre-deoxygenated solid BHI medium), placing into an anaerobic box and culturing at 37° C. for 72 h; picking a single clone and inoculating it onto a new BHI solid plate for subculture.

### (3) Strain Identification

Picking a colony with a small pipette tip into a 1.5 ml centrifuge manifold, adding 20 μL double distilled water and heating in a boiling water bath for 5 min. Then performing colony PCR using 16S rDNA universal primers.

The primer sequences used are:

| Name | Sequence |
|---|---|
| F340 | ACTCCTACGGGAGGCAGCAGT (SEQ ID NO: 1) |
| R1492 | GGTTACCTTGTTACGACTT (SEQ ID NO: 2) |

The PCR reaction system is:

| Components | Volume (μL) |
|---|---|
| 2 × taq MIX | 10 |
| Upstream and downstream primers, each 10 μM | 1 |
| dd H₂O | 8 |
| Colony | 1 |
| Total volume | 20 |

The reaction procedures are:

| Step | Temperature (° C.) | Time (sec) |
|---|---|---|
| Predenaturation | 95 | 300 |
| Denaturation | 95 | 20 |
| Annealing | 58 | 30 |
| Extending | 72 | 60 |
| Back to denaturation step | — | — |
| Cycle for 30 times | | |
| Continue extending | 72 | 300 |
| Cooling | 4 | Hold |

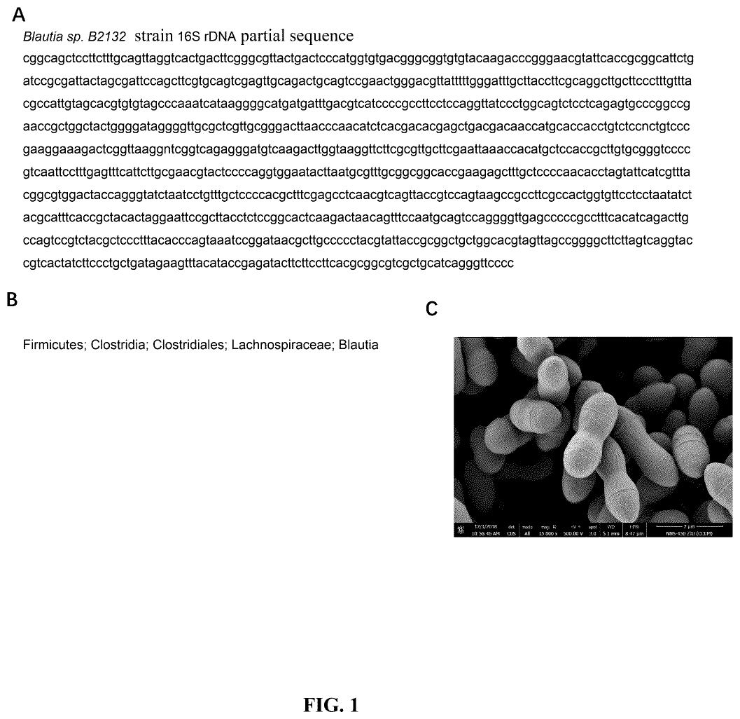

The PCR products were then sent to a biotech company for sequencing. The sequence was shown in FIG. 1A, and compared in the RDP database to obtain the species classification information, from which it can be confirmed as a Blautia sp. species, as shown in FIG. 1B.

The obtained Blautia sp. B2132 strain is preserved, of which the preservation information is as follows: the preservation name is Blautia sp., Depository Authority: Common Microorganism Center of China Committee for Culture Collection of Microorganisms, Preservation address: No. 3, Courtyard 1, Beichen West Road, Chaoyang District, Beijing; Deposit No: CGMCC NO. 1.5296, Preservation time: Oct. 8, 2019.

### (4) Observation on the Morphology of Bacteria Through a Scanning Electron Microscope

First, an inoculated loop of colonies were selected in the anaerobic incubator and placed into 10 ml of pre-deoxygenated BHI liquid medium, and then placed in the anaerobic incubator at 37° C., and cultured overnight while standing to make the bacteria liquid turbid. Then, 1.5 ml of bacterial sample was taken and centrifuged at 4000 rpm for 5 min, the supernatant was discarded, and then washed once with PBS buffer. The samples were then resuspended with 1.5 ml of the fresh formulated glutaraldehyde (formulated with PBS buffer at PH=7.4) at a volume concentration of 2.5%, and fixed overnight at 4° C. After the fixation, the cells were collected by centrifugation at 4000 rpm for 5 min, the glutaraldehyde fixative was discarded, and the samples were washed 3 times with PBS buffer for 15 min each, and then 100 μl of 1% (mass %) osmic acid solution was added to fix the samples at room temperature for 1 h. Then the cells were collected by centrifugation at 4000 rpm for 5 min, the osmic acid fixative was discarded, and the samples were washed 3 times with PBS buffer for 15 min each. Then the samples were dehydrated with ethanol at a volume concentration gradient of 30%, 50%, 70%, 80%, 90%, 95%, 100% respectively, for 15 min for each concentration. Finally, the samples were dried at critical point (a routine operation step in sample preparation for scanning electron microscope), and the samples were observed through a scanning electron microscope. The bacteria morphology observed through the scanning electron microscope was shown in FIG. 1C.

In summary, monoclonal strains were isolated from the feces of healthy mice, and the bacteria were identified through 16S rDNA sequence alignment as species of Bacteria, Firmicutes, Clostridia, Clostridiales, Lachnospiraceae, Blautia sp; the scanning electron microscope image shows that Blautia sp. B2132 strain is similar to a dumbbell, with circular patterns and no flagella.

### Embodiment 2. Abundance Setection of Blautia Bacteria Content in IBD Patients and Healthy Controls

### (1) Fecal Flora DNA Extraction

Fecal samples of IBD patients were provided by inpatients at the Inflammatory Bowel Disease Center of Sir Run Run Shaw Hospital, Zhejiang University. After the samples were collected, they were frozen and stored in a refrigerator at −20° C. in time, and transported while being frozen on dry ice to a refrigerator at −80° C. in the laboratory within 2 days for preservation. Fecal samples of healthy people were provided by healthy physical examinees, and taken back to the laboratory and stored in a refrigerator at −80° C. within 5 hours. When extracting fecal DNA, the samples were taken out from −80° C., from which 100-150 mg of feces was taken with a medicine spoon into a 1.5 ml centrifuge tube without thawing; then, a fecal flora DNA extraction kit (Qiagen) was employed to extract the total fecal flora DNA, and then the concentration, purity and quality of DNA were detected using a Nanodrop 2000 ultraviolet microspectrophotometer and agarose gel electrophoresis, and the follow-up operations were performed after passing the quality inspection.

### (2) Detection of the Content of Blautia Sp. Strain in the Samples by the Quantitative PCR Method

The above fecal flora DNA was diluted to 10 ng/ul, then added into a 384-well plate, and detected quantitatively with a Roche real-time quantitative PCR instrument.

The primer sequences are:

| Name | Sequence |
|---|---|
| Universal | ACTCCTACGGGAGGCAGCAGT |
| bacterial F | (SEQ ID NO: 3) |
| Universal | ATTACCGCGGCTGCTGGC |
| bacterial R | (SEQ ID NO: 4) |
| Blautia F | AGGAAGAAGTATCTCGGTAT (SEQ ID NO: 5) |
| Blautia R | ATCAGACTTGCCACACCGTC (SEQ ID NO: 6) |

The PCR reaction system is:

| Components | Volume (μL) |
|---|---|
| 2 × SYBR Green | 3 |
| Upstream and downstream primers (Blautia), | |
| each 1 μM | 1 |
| dd H₂O | 1 |
| DNA | 1 |
| Total volume | 6 |

The reaction procedures are:

| Step | Temperature (° C.) | Time (sec) |
|---|---|---|
| Denaturation | 95 | 30 |
| Cycling | 95 | 5 |
| (45 times) | 60 | 30 |
| Cooling | 50 | 15 |

All the samples were subjected to three parallel tests. With Universal bacteria as the internal reference, the relative abundance of Blautia sp. strain in each sample was calculated by the 2⁻^{^{ΔΔ}}^{Ct} method.

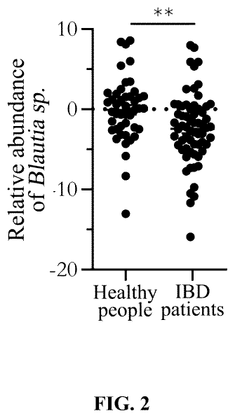

The results were shown in FIG. 2. It can be known from FIG. 2 that, the imbalance of intestinal flora is closely related to the occurrence and development of IBD, and the content of Blautia sp. strain in the intestine of IBD patients is significantly lower than that of healthy people.

### Embodiment 3. Effect of the Abundance of Blautia Sp. Strain in the Intestinal Flora on Spontaneous Enteritis in Il-10^{-/-} Mice

Immune cells of Il-10 knockout mice (Il-10^{-/-} mice) cannot produce Interleukin 10 (IL-10). Therefore, under normal feeding conditions, Il-10^{-/-} mice around 12 weeks of age will develop spontaneous enteritis, especially inflammation of the colon, which is characterized by inflammatory infiltration of lymphocytes, macrophages and neutrophils. The severity of the disease is closely related to the structural composition of the intestinal flora. Therefore, the present disclosure uses the Il-10^{-/-} mice spontaneous enteritis model to evaluate the relationship between Blautia sp. strain and the onset of enteritis.

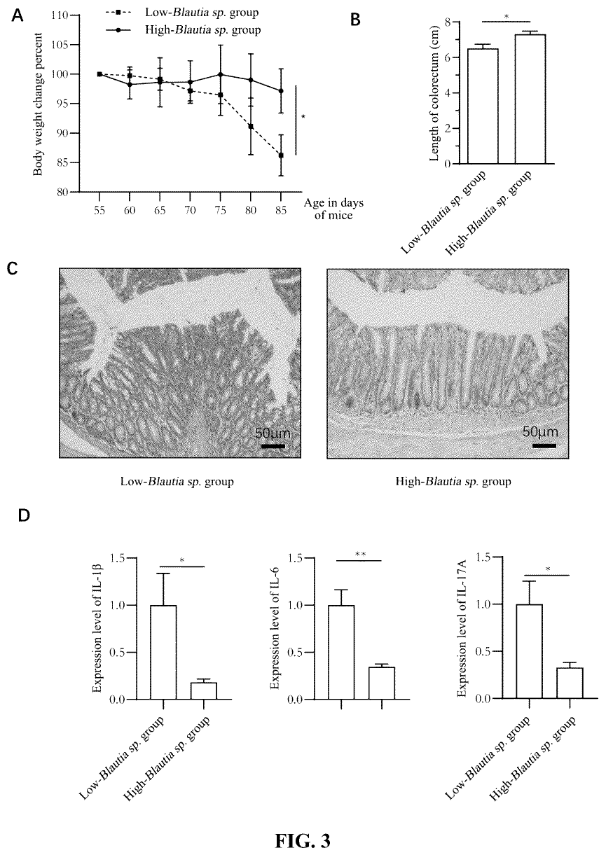

First, 10 sex and age matched Il-10^{-/-} mice were kept in an SPF environment for 8 weeks. Then, the relative abundance of Blautia sp. strain in the intestinal flora of the 10 Il-10^{-/-} mice was detected by the quantitative PCR method, and the mice were ranked according to the abundance of Blautia sp. strain from high to low, from which 5 mice with high abundance were taken as a high-Blautia sp. strain group, and 5 mice with low abundance were taken as a low-Blautia sp. strain group. After grouping, the mice were fed continually for 4 weeks, and the weight of mice was recorded every 5 days. The results showed that the weight loss of the Il-10^{-/-} mice in the high-Blautia sp. strain group was significantly less than that of the low-Blautia sp. strain group (as shown in FIG. 3A). After feeding to the 12th week (85 days of age), the mice were dislocated from the cervical vertebra, and the length of the colon was measured. The results showed that the intestine of the Il-10^{-/-} mice in the high-Blautia sp. strain group was significantly longer than that in the low-Blautia sp. strain group (as shown in FIG. 3B). Then, the histomorphology of the intestinal tissue was analyzed by H&E staining analysis, showing that the histomorphology of the Il-10^{-/-} mice in the high-Blautia sp. strain group was more normal, and the inflammatory cell infiltration was lower than that in the low-Blautia sp. strain group (as shown in FIG. 3C). The expression of inflammatory factors in mice was analyzed by the quantitative PCR method, with the results showing that the expression of inflammatory factors in the Il-10^{-/-} mice in the high-Blautia sp. strain group was significantly longer than that in the low-Blautia sp. strain group (as shown in FIG. 3D).

According to FIG. 3, it shows that the content of Blautia sp. strain in the intestinal flora is inversely proportional to the degree of development of enteritis in the Il-10^{-/-} mice. That is, high content of Blautia sp. strain can inhibit the development of enteritis in the Il-10/-mice, while low content of Blautia sp. strain can aggravate the development of enteritis in the Il-10^{-/-} mice.

### Embodiment 4. Cultivation of Blautia Sp. B2132 Strain and Detection of Butyric Acid Production Capacity

### (1) Culture of Blautia Sp. B2132 Strain

The Blautia sp. B2132 strain, with a deposit number of CGMCC NO. 1.5296, was resuscitated in an anaerobic incubator. After anaerobic cultivation at 37° C. for 48 hours, the colony has grown to a diameter of 1-2 mm. Then one colony was picked with a pipette into 15 ml pre-deoxygenated liquid BHI medium, and anaerobically cultured at 37° C. for 48 hours while standing, to obtain the Blautia sp. B2132 strain solution, of which the density was about 7×10⁸ CFU/mL.

### (2) Detection of Butyric Acid Production Capacity of Blautia Sp. B2132 Strain

1 ml of the above Blautia sp. B2132 strain solution was taken and centrifuged at 12000 r/min for 5 min. The supernatant was taken, diluted 1000 times with double distilled water, and adjusted to pH 2-3 with hydrochloric acid solution, and mixed well by vortex shaking; and then centrifuged at 12000 r/min for 10 min. The supernatant was collected carefully into a chromatographic bottle. At the same time, standard butyric acid solutions of 10 μg/mL, 50 μg/mL, 100 μg/mL, 200 μg/mL, 500 μg/mL and 1000 μg/mL were formulated. After then, the samples were analyzed using Agilent 6890N gas chromatograph in conjuction with a gas chromatographic column DB-624UI: 1 μL of sample was taken into the injection hole, the initial column temperature was 100° C., the carrier gas was high-purity nitrogen, the flow rate was 1.1 mL/min, holding for 3 minutes; then the temperature was elevated to 200° C. at a rate of 10° C./min for 3 min; finally, a standard curve was drawn based on the area under the curve of standard butyric acid solution, and the concentration of butyric acid in the strain solution was calculated based on the area under the curve of the sample butyric acid. It was determined that under the condition of pre-deoxygenated BHI medium, the butyric acid production of Blautia sp. B2132 strain at 48 hour was 39.7 mmol/L.

### Embodiment 5. Blautia sp. B2132 Strain Inhibits the Growth of Harmful Intestinal Bacteria α-Proteobacteria

### (1) Preparation of Wild-Type Mice

14 8-week-old wild-type BL/C56 mice were firstly purchased from Shanghai SLAC Co. and kept in the SPF barrier system of the Laboratory Animal Center of Zhejiang University. After adaptation for one week, the mice were randomly divided into a BHI solvent control group and a Blautia sp. B2132 strain treatment group, with 7 mice in each group.

### (2) Intestinal Colonization of Blautia Sp. B2132 Strain

The Blautia sp. B2132 strain solution cultured in Embodiment 4 was taken and centrifuged at 4000 rpm for 5 min to collect the bacterial cells. The bacteria were then resuspended in pre-deoxygenated liquid BHI medium to a final concentration of about 10⁹ CFU/ml. WT mice were treated by intragastric administration at a dose of 200 μL Blautia sp. B2132 per mouse every other day for 2 weeks. At the same time, the control group was only gavaged with the pre-deoxygenated liquid BHI medium.

### (3) Quantitative Detection of the Abundance of Blautia Sp. B2132 Strain and the Abundance of α-Proteobacteria

On day 7 after the completion of the gavage treatment, the feces of mice was collected, the fecal flora DNA was extracted according to the method described in the above Embodiment 2 and detected by the quantitative PCR.

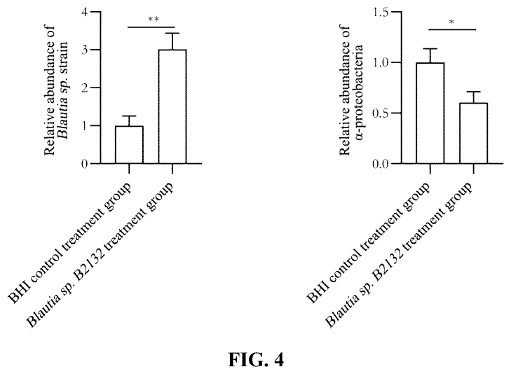

The results were shown in FIG. 4, from which it can be concluded that, after intragastric supplementation of Blautia sp. B2132 strain, the abundance of the harmful intestinal bacteria α-proteobacteria was significantly reduced, that was, Blautia sp. B2132 strain could regulate the intestinal flora.

### Embodiment 6. Oral Supplementation of Blautia Sp. B2132 Strain Can Alleviate the Phenotype of Spontaneous Enteritis in Il-10^{-/-} Mice

### (1) Intestinal Colonization of Blautia sp. B2132 Strain

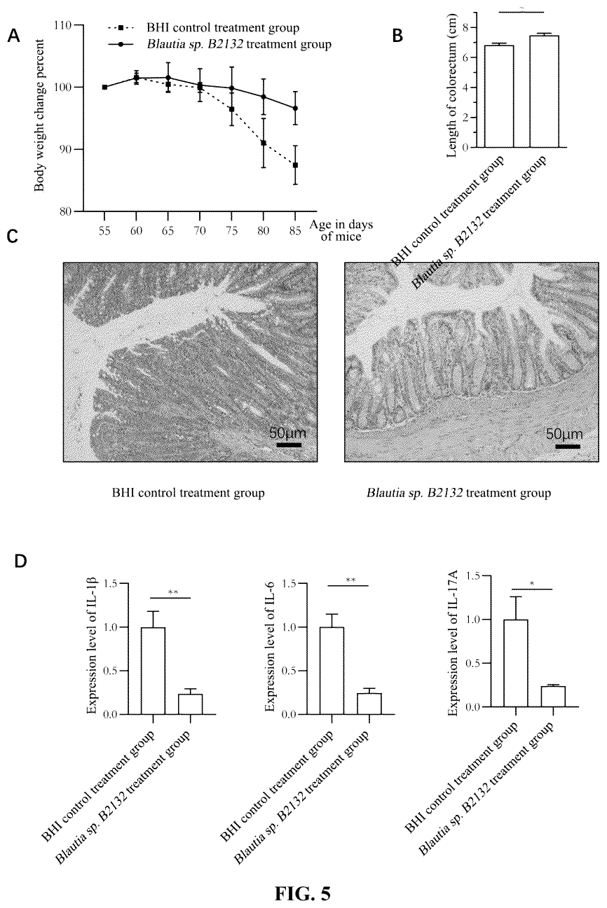

16 6-week-old Il-10^{-/-} mice were randomly divided into 2 groups evenly, a Blautia sp. B2132 treatment group and a culture solution BHI control treatment group, respectively. The specific treatment was as follows: for the Blautia sp. B2132 treatment group, the Blautia sp. B2132 strain solution cultured in Embodiment 4 was firstly taken and centrifuged at 4000 rpm for 5 min to collect the bacterial cells; the bacteria were then resuspended in pre-deoxygenated liquid BHI medium to a final concentration of about 10⁹ CFU/ml; the Il-10^{-/-} mice were treated by intragastric administration at a dose of 200 μL Blautia sp. B2132 per mouse every other day for 2 weeks. For the culture solution BHI control treatment group, the mice were treated by intragastric administration with 200 μL pre-deoxygenated liquid BHI medium every other day for 2 weeks. The mice were raised to 12 weeks of age (85 days of age), the body weight of mice was measured. The results showed that the weight loss of mice in the Blautia sp. B2132 treatment group was significantly less than that of the BHI control treatment group (as shown in FIG. 5A). At the 12th week (85 days of age), the mice were dislocated from the cervical vertebra, and the colon was taken out of mice and measured for its length, with the results showing that the intestine of mice in the Blautia sp. B2132 treatment group was significantly longer than that in the BHI control treatment group (as shown in FIG. 5B); about 1 cm of distal colon tissue segment was taken for Formalin fixation, and subsequently subjected to HE staining and histomorphological analysis, with the results showing that the inflammatory cell infiltration of mice in the Blautia sp. B2132 treatment group was significantly less than that in the BHI control treatment group (as shown in FIG. 5C); about 1 cm of intestinal segment was taken to extract RNA, which was detected by the fluorescence quantitative PCR method for the expression of inflammatory factors, with the results showing that the expression of inflammatory factors of mice in the Blautia sp. B2132 treatment group was significantly lower than that in the BHI control treatment group (as shown in FIG. 5D).

According to FIG. 5, it can be known that, in Il-10^{-/-} mice spontaneous enteritis model, the supplementation of Blautia sp. B2132 can improve the severity of spontaneous enteritis.

Finally, it should be noted that the above-listed are only a few specific embodiments of the present disclosure. Obviously, the present disclosure is not limited to the above embodiments, and many variations are possible. All variations that can be directly derived or associated by a person of ordinary skills in the art from the content of the present disclosure should be regarded as the protection scope of the present disclosure.

1. A Blautia sp. B2132 strain, wherein: the Blautia sp. B2132 strain has a deposit number of CGMCC NO. 1.5296.

2. An application of the Blautia sp. B2132 strain according to claim 1 in the preparation of a medicine for regulating the intestinal flora and preventing and/or treating an inflammatory bowel disease.

3. The application according to claim 2, wherein: the medicine comprises a pharmaceutically effective dose of Blautia sp. B2132 strain and a pharmaceutically acceptable carrier.

4. The application according to claim 3, wherein: the pharmaceutically effective dose is 10⁶-10¹⁰ CFU.

5. The application according to claim 3, wherein, the pharmaceutically acceptable carrier is milk powder, lactose, cyclodextrin, maltose, glucose, glycerin, sodium glutamate, vitamin C, mannose, galactose, mannitol or methylcellulose.

6. The application according to claim 1, wherein: the inflammatory bowel disease is ulcerative colitis or Crohn's disease.

7. A pharmaceutical composition for regulating the intestinal flora, preventing and/or treating an inflammatory bowel disease, wherein, the pharmaceutical composition comprises a pharmaceutically effective dose of Blautia sp. B2132 strain with a deposit number of CGMCC No. 1.5296.

8. The pharmaceutical composition according to claim 7, wherein: the pharmaceutically effective dose is 10⁶-10¹⁰ CFU.

9. A food/health care product/food additive for preventing and/or treating an inflammatory bowel disease, wherein, the food/health care product/food additive comprises a Blautia sp. B2132 strain with a deposit number of CGMCC No. 1.5296.

10. The application according to claim 4, wherein, the pharmaceutically acceptable carrier is milk powder, lactose, cyclodextrin, maltose, glucose, glycerin, sodium glutamate, vitamin C, mannose, galactose, mannitol or methylcellulose.

11. The application according to claim 2, wherein: the inflammatory bowel disease is ulcerative colitis or Crohn's disease.

12. The application according to claim 3, wherein: the inflammatory bowel disease is ulcerative colitis or Crohn's disease.

13. The application according to claim 4, wherein: the inflammatory bowel disease is ulcerative colitis or Crohn's disease.

14. The application according to claim 5, wherein: the inflammatory bowel disease is ulcerative colitis or Crohn's disease.


SEQUENCE LISTING (6 sequences)

SEQ ID NO: 1 - DNA, 21 bp, Artificial Sequence
  universal primers
actcctacgggaggcagcagt

SEQ ID NO: 2 - DNA, 19 bp, Artificial Sequence
  universal primers
ggttaccttgttacgactt

SEQ ID NO: 3 - DNA, 21 bp, Artificial Sequence
  primers
actcctacgggaggcagcagt

SEQ ID NO: 4 - DNA, 18 bp, Artificial Sequence
  primers
attaccgcggctgctggc

SEQ ID NO: 5 - DNA, 20 bp, Artificial Sequence
  primers
aggaagaagtatctcggtat

SEQ ID NO: 6 - DNA, 20 bp, Artificial Sequence
  primers
atcagacttgccacaccgtc